(19) United States
(12) Patent Application Publication
(10) Pub. No.: US 2025/0049623 A1
(43) Pub. Date: Feb. 13, 2025

(54) BIODEGRADABLE PATIENT LATERIAL TRANSFER SUPPORT SURFACE AND METHOD OF MAKING

(71) Applicant: Bannack Medical LLC, Gilbert, AZ (US)

(72) Inventors: Arlen Olsen, Gilbert, AZ (US); Richard Mayer, Chandler, AZ (US)

(21) Appl. No.: 18/490,439

(22) Filed: Oct. 19, 2023

Related U.S. Application Data
(60) Provisional application No. 63/531,639, filed on Aug. 9, 2023.

(51) Int. Cl. A61G 7/10; A61L 31/04; A61L 31/12; A61L 31/16

(52) U.S. Cl. CPC A61G 7/1028; A61L 31/041; A61L 31/129; A61L 31/16; A61L 2300/404

(57) ABSTRACT

The disclosed relates to a lateral transfer support surface and method of making. The flexible material sheets are sealed to one another to form at least one chamber, at least one of the flexible material sheets having air exit holes therein. The flexible material sheets is made of a polymer selected from a group consisting of polyamide, polyester, polyethylene (PE), polypropylene (PP), polyvinylchloride (PVC) and combinations thereof; and an additive comprising a blend or copolymer of (1) a first polymer selected from a poly-lactic acid (PLA), a polyhydroxyalkanoates (PHA) or a combination thereof and (2) a second polymer which is a poly-terephthalate.

This application claims priority to U.S. provisional patent application Ser. No. 63/531,639 filed on Aug. 9, 2023, the entirety of which is hereby incorporated by reference.

### TECHNICAL FIELD

The following relates to a biodegradable patient support surfaces a method of making. In particular, the invention is a biodegradable single patient lateral transfer support surface, patient lifting and patient slings most often, for use in health care, to maintain a clean transfer surface due to body fluids sustained from injury or infectious diseases, a method and composition.

### BACKGROUND

Disposal of plastic waste is a serious environmental problem. Increased use of plastics and other synthetic materials has resulted in a growing environmental impact. To combat this, bioplastics such as biodegradable polymers are being developed to be used as an alternative for non-biodegradable polymer materials. The best option for managing non-biodegradable plastic waste is to replace non-biodegradable materials with biodegradable polymers as they are environmentally friendly. Alternatively, non-biodegradable plastics may be recycled. Many of the products that hospitals and healthcare providers use are disposable plastic products that are non-biodegradable. This is a problem and a growing concern for hospitals and healthcare facilities meeting sustainability goals.

Recently, healthcare providers are moving away from traditional woven cloth materials due to their retention of infectious disease due to their porosity. Instead, they are moving toward a hospital disposable, such as a single use patient lateral transfer support surface. With increased demand for disposable single-patient lateral transfer support surfaces (mattresses), this creates a problem for being able to dispose of the single patient supports surfaces after use. Many solutions are being considered other than placing them in landfills, such as recycling by melting the support surface down. This creates the challenge of having the healthcare workers obtaining adequate recycling solutions. Many hospitals and healthcare workers are also under pressure to come up with sustainability solutions for hospital waste.

Examples of single use lateral transfer patient support surfaces at the following locations Examples of patents include U.S. Pat. Nos. 6,898,809; 7,712,170 and 7,373,680 which are herby incorporated by reference.

### SUMMARY

An aspect is to make a biodegradable patient lateral transfer support surface.

Another aspect is to have an additive material that can make most polymeric compositions biodegradable by merely mixing it in with the polymeric material any time before the polymeric material is formed into an article.

Another aspect is to provide a method for preparing a biodegradable single patient lateral transfer support surface.

A further aspect is to provide a composition of biodegradable single patient lateral transfer support surface.

A further aspect is to provide a composition of biodegradable buckles used in single patient lateral transfer support surface.

One embodiment of the present invention relates to a lateral transfer support surface, comprising: flexible material sheets sealed to one another to form at least one chamber, at least one of the flexible material sheets having air exit holes therein, said flexible material sheets comprised of: a polymer selected from a group consisting of polyamide, polyester, polyethylene (PE), polypropylene (PP), polyvinylchloride (PVC) and combinations thereof; and an additive comprising a blend or copolymer of (1) a first polymer selected from a poly-lactic acid (PLA), a polyhydroxyalkanoates (PHA) or a combination thereof and (2) a second polymer which is a poly-terephthalate.

In another embodiment of the present invention relates to a biodegradable lateral transfer support surface comprising: a. fabric; and b. hardware, wherein the fabric and hardware are both made from a material comprised of: a polymer selected from a group consisting of polyamide (characterized by the presence of amide groups (CO—NH) in the main polymer chain), polyester, polyethylene (PE), polypropylene (PP), polyvinylchloride (PVC) and combinations thereof; and a biodegradable additive, wherein the fabric has a first hardness on the durometer scale and the hardware has a second hardness on the durometer scale, different than the first hardness on the durometer scale.

In another embodiment of the present invention relates to a biodegradable lateral transfer support surface or fabric material comprising: a main body selected from polyamide or polyester; b. inflatable chambers comprise a polymer selected from a group consisting of polyamide, polyester, polyethylene (PE), polypropylene (PP), polyvinylchloride (PVC) and combinations thereof; c. straps and handles selected from a group consisting of nylon (polyamide), polyester, polyethylene (PE), polypropylene (PP), polyvinylchloride (PVC) and combinations thereof; d. fasteners selected from a group consisting of nylon, polyester, polyethylene (PE), polypropylene (PP), polyvinylchloride (PVC) and combinations thereof; and e. an additive added to each of the materials in a-d comprising a blend or copolymer of (1) a first polymer selected from a poly-lactic acid (PLA), a polyhydroxyalkanoates (PHA) or a combination thereof and (2) a second polymer which is a poly-terephthalate. The fabric or fabric material may be coated with a polyurethane and/or water repellant coating mixed with a liquid additive added to polyurethane and/or water repellant coating comprising a blend or copolymer of (1) a first polymer selected from a poly-lactic acid (PLA), a polyhydroxyalkanoates (PHA) or a combination thereof and (2) a second polymer which is a poly-terephthalate.

Another embodiment of the present invention includes adding an antimicrobial to the manufacturing process. The most common additives used to manufacture antimicrobial plastics include various isothiazolinone treatments, zinc pyrithione, thiabendazole, and silver antimicrobial products.

The further embodiment of the present invention provides a method for preparation of biodegradable lateral transfer support surface.

### BRIEF DESCRIPTION OF THE DRAWINGS

The following disclosure as a whole may be best understood by reference to the provided detailed description when read in conjunction with the accompanying drawings, drawing description, abstract, background, field of the disclosure, and associated headings. Identical reference numerals when found on different figures identify the same elements or a functionally equivalent element. The elements listed in the abstract are not referenced but nevertheless refer by association to the elements of the detailed description and associated disclosure.

FIG. 1 illustrates a biodegradable lateral transfer support surface according to the present disclosure;

FIG. 2 illustrates a sectional view of the biodegradable lateral transfer support surface; and

FIG. 3 illustrates the cross-sectional view showing a baffle.

### DETAILED DESCRIPTION

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness.

An embodiment of the biodegradable lateral transfer support surface (1) of the present invention is shown in FIG. 1. The lateral transfer support surface is generally single use or single patient use. After use the surface is disposed of. According to the system of the present invention, a body pad, mattress or support surface made of flexible material sheets to form at least one chamber (2) in FIG. 1 is assembled with an air supply system (11). The supply system (11) has included a gas/air pump (8), an expandable gas/air hose (7) and HEPA Filter (9). A power supply with a power cord (10) is provided. The cord (10) can be plugged into an AC outlet for running the air pump (8) which provides continuous forced air to body pad. The expandable hose (7) has a quick connect valve (6) on a distal end for connection to the body pad (2). In an alternate embodiment of the invention, body pad (2) is provided with safety straps (3), hook loop fasteners (4) and buckles (31). For further stabilization of the patient, head support (21) and positionary handles (5) may be included, attached to the body pad (2).

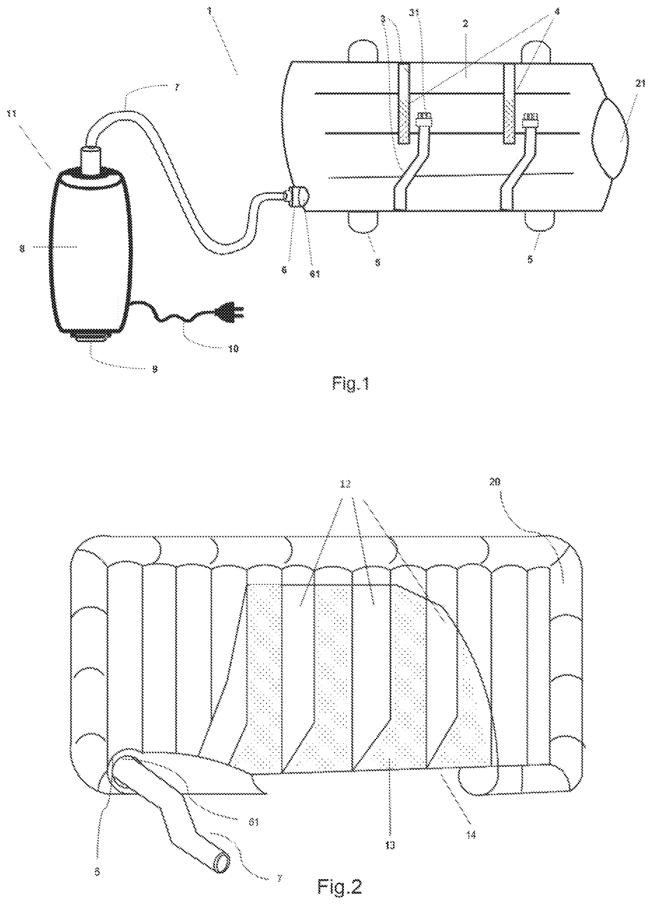

FIG. 2 illustrate a perspective view of the transfer mattress (20) including a bottom panel (14) that may include a plurality of air exit holes (13) configured to provide an air cushion under the mattress (20) when the mattress (20) is inflated or pressurized. When the mattress (2) is pressurized, air can escape under pressure through the pinholes or air exit holes (13) and acts initially to jack a load placed upon the mattress (2) above the underlying support surface. The top, bottom and/or both chambers are ridged and/or contain baffle panels (12) to enhance dimensional stability to the device. The supply system (11) has included a gas/air supply hose (7) which has a quick connect (6) on the inlet opening (61) of the air mattress (20). The bottom panel (14) is sealed to the side pnels, top panels and baffles to make it airtight with the exception of the pinholes or air exit holes (13).

Many mattresses are considered a closed system wherein a pump would pressurize the mattress and be turned off and the mattress would remain inflated. In this case, it is not a closed system, but an open system requiring a constant flow gas/air pump (8), that continually pumps air while in use while air is lost through the pinholes or air exit holes (13). The purpose of the mattress (20) is to transfer or lift the patient for a short period of time and shut the pump (8) off after transfer. For example, a user will roll or place a patient on the mattress (20), the pump (8) will be turned on, the patient will be secured or strapped into the mattress (20) with safety straps (3), hook loop fasteners (4) and buckles (31), the transfer will occur while maintaining constant air flow (8) and the patient will then be removed from the mattress (20). Generally, the inside surface is coated with a biodegradable polyurethane and the outside surface is covered with a biodegradable water-proof coating.

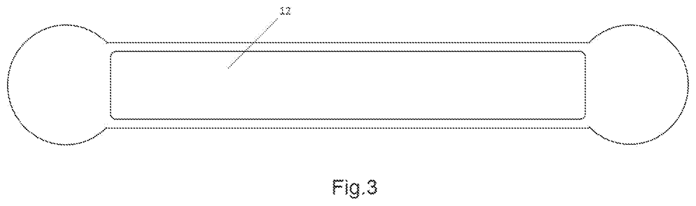

FIG. 3 illustrate a cross-sectional view, showing a baffle (12) and a dimensional relationship of a radially-outwardly curved perimeter wall to the mattress as a whole.

In further embodiment, the present invention relates to a composition of matter comprising: a non-biodegradable polymer selected from a group consisting of nylon (polyimide), polyester, polyethylene (PE), polypropylene (PP), polyvinylchloride (PVC) and combinations thereof; an additive comprising a blend or copolymer of (1) a first polymer selected from a poly-lactic acid (PLA), a polyhydroxyalkanoates (PHA) or a combination thereof and (2) a second polymer which is a poly-terephthalate; and wherein the non-biodegradable polymer is present in a concentration from 90-99.9 wt. %, the additive is present in a concentration from 0.1-10 wt. % and the additive has 30-70 wt. % of the first polymer and 30-70% of the second polymer.

The another embodiment of the present invention is a composition of biodegradable buckles: a polymer i.e. polypropylene (PP); an additive comprising a blend or copolymer of (1) a first polymer selected from a poly-lactic acid (PLA), a polyhydroxyalkanoates (PHA) or a combination thereof and (2) a second polymer which is a poly-terephthalate; and wherein the non-biodegradable polymer is present in a concentration from 90-99.9 wt. %, the additive is present in a concentration from 0.1-10 wt. % and the additive has 30-70 wt. % of the first polymer and 30-70% of the second polymer.

### Biodegradable Additive

In one embodiment, the first polymer and second polymer of additive are covalently bound to one another to form a copolymer. In another embodiment, the first polymer and second polymer are blended together to form an admixture but are not covalently bound to one another.

The first polymer is a PLA, a PHA or a combination thereof. The term PLA includes poly-D-lactic acid (PDLA), poly-L-lactic acid (PLLA) and combinations thereof. Specific examples of PHAs include poly-3-hydroxybutyrate (PHB), poly-3-hydroxybutyrate-co-4-hydroxybutyrate (P(3-HB-co-4-HB)), poly-3-hydroxybutyrate-co-valerate (PHBV), and polyhydroxybutyrate-co-hexanoate (PHBH). In one embodiment, the first polymer is PLA sold under the brand name INGEO®and has a number-average molecular weight of 127 kg per mole and a polydispersity index of 1.6. In other embodiments, the number-average molecular weight is between 100-150 kg per mole.

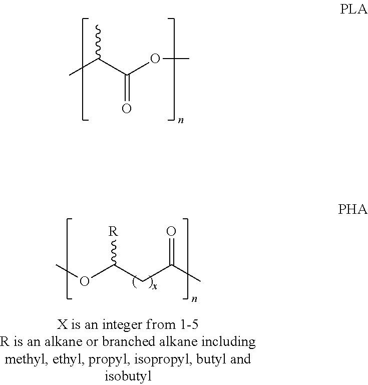

The second polymer is a poly-terephthalate. Examples include copolymers such as polybutylene adipate terephthalate (PBAT), polybutylene terephthalate polycyclohexylenedimethylene terephthalate, polyethylene terephthalate, polytrimethylene terephthalate, poly(butylene succinate terephthalate) (PBST) and poly(butylene sebacate terephthalate). In one embodiment, the second polymer is a PBAT polymer sold under the brand name ECOFLEX® and has a number-average molecular weight of about 52 kg per mole and a polydispersity index of 2. In other embodiments, the number-average molecular weight is between 40 and 60 kg per mole.

In one embodiment, the additive comprises a blend of 30-70 wt. % of the first polymer and 30-70 wt. % of the second polymer. In another embodiment, the additive comprises a blend of 40-60 wt. % of the first polymer and 40-60 wt. % of the second polymer. In another embodiment, the additive comprises a blend of 45-55 wt. % of the first polymer and 45-55 wt. % of the second polymer. In another embodiment, the additive comprises a blend of 49-51 wt. % of the first polymer and 49-51 wt. % of the second polymer. In yet another embodiment, the additive consists of a blend of 49-51 wt. % of the first polymer and 49-51 wt. % of the second polymer.

In one embodiment, the second polymer is a blend/admixture of a polyester (1) and a poly-terephthalate (2), the structure of which are shown below. In another embodiment, the second polymer is a copolymer prepared by transesterifying polyester (1) and poly-terephthalate (2). The values of a, b and c are integers independently selected from 1-8. In one embodiment, a and c are integers from 1-6 and b is an integer from 1-8. In one embodiment, a and c are both 4. In one embodiment, b is an integer from 1-4. In another embodiment, b is an integer from 5-8. In one embodiment, the values of a, b and c are all four. The transesterification is performed using conventional methods including acid catalyzed transesterification. Organometallic catalysts are known for facilitating such a reaction including tetrabutoxytitanium and other zinc, tin and germanium-based catalysts. The tranesterification may be performed at high temperature (e.g. greater than 190° C.) and under vacuum to facilitate removal of volatile byproducts, including water. During transesterification, random block copolymerization of the monomeric units may occur.

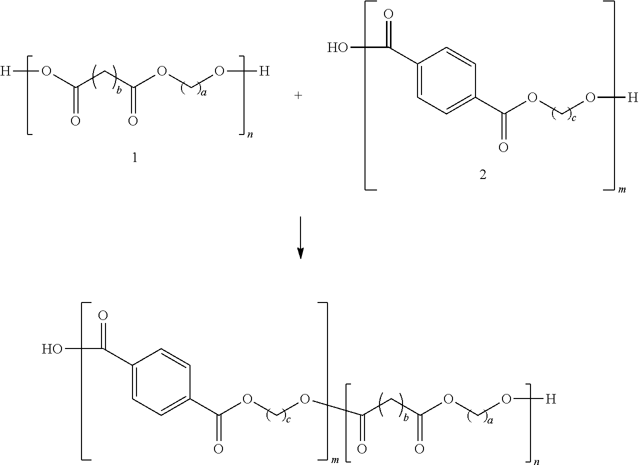

In one embodiment, a=b=c=4 and the poly-terephthalate (2) is polybutylene adipate terephthalate (PBAT).

In one embodiment, the values of m and n are within 30% of one another (e.g. a m:nmolar ratio of 1.3:1 to a ratio of 1:1.3. In another embodiment, the values of m and n are within 20% of one another (e.g. a m:n ratio of 1.2:1 to 1:1.2). In another embodiment, the values of m and n are within 10% of one another (e.g. a m:n ratio of 1.1:1 to 1:1.1). In another embodiment, the values of m and n are within 5% of one another (e.g. a m:n ratio of 1.05:1 to 1:1.05). In another embodiment, the values of m and n are within 1% of one another (e.g. a m:n ratio of 1.01:1 to 1:1.01). In one embodiment, the terephthalate monomer is present in the additive at a concentration of less than 55 mol %.

The polyester (1) may be prepared from an alkane diol (3) and a diacid (R₁=H) (4). In one embodiment, R₁ in the diacid (4) is methyl, ethyl or propylsuch that acorresponding diester is used. In one embodiment, a is four such that compound (3) is 1,4-butane diol. In some embodiments, R₁ is hydrogen and b is four (adipic acid), two (succinic acid) or eight (sebacic acid).

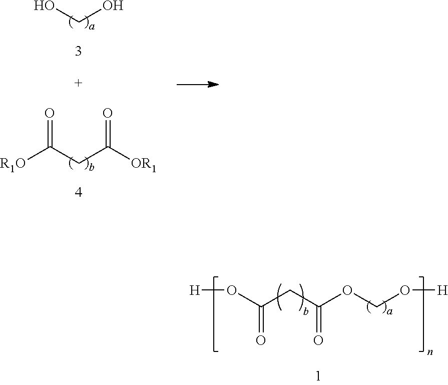

In one embodiment, R₁ is hydrogen and b is four (i.e. adipic acid) and a is four (i.e. 1,4-butane diol) as shown below.

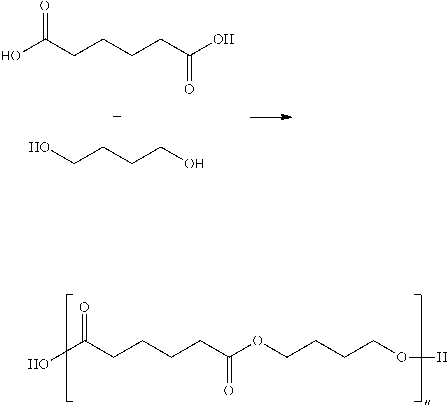

The poly-terephthalate (2) is prepared from a terephthalate ester (5) (or its corresponding acid) and alkane diol (6). In terephthalate ester (5) R₁ may be H or an alkane such as methyl, ethyl, propyl, isopropyl, etc. The value of c is an integer from 1-6. In one embodiment, c is four such that compound (6) is 1,4-butane diol.

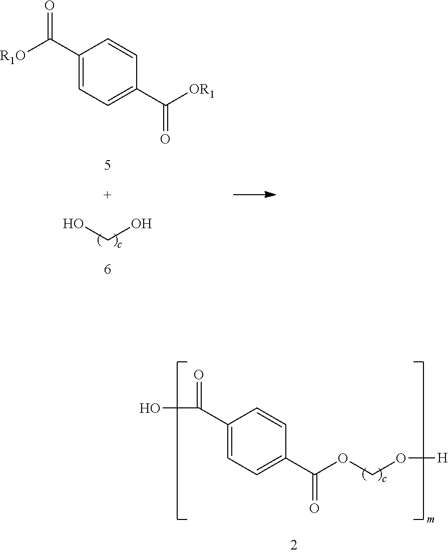

In one embodiment, R₁ is methyl and c is four, as shown below.

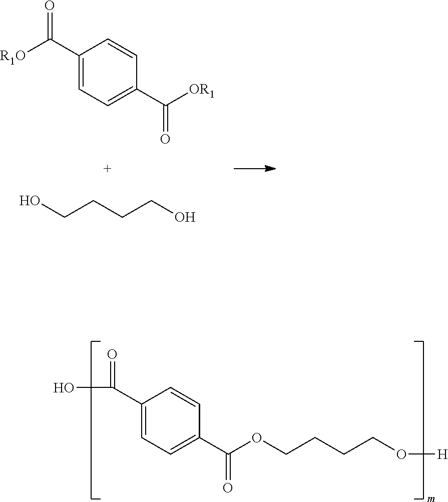

The additive has a crosslinking density that renders it biodegradable. In one embodiment, the crosslinking density is less than 30%, less than 20% or less than 10%. The crosslinking density can be determined by using ASTM-D2765. This method determines gel content and swell ratio for a known mass of polymer extracted with a suitable solvent. The extracted material is separated from the solvent and weighed after drying. The higher the mass of extracted material, the lower the crosslinking density.

The additive has a glass transition temperature (T_{g}) within or below the optimal temperature of mesophilic bacteria (e.g. a T_{g} within a temperature range of 20° C. to 45° C.).

The further embodiment of the invention discloses that 1% additive will be added to each material used to manufacture the lateral transfer support surface including; For example, the Nylon or Polyester Fabric; PVC air chambers; nylon webbing or reinforced plastic of the straps and handles; the Buckles and Fasteners which can be a polypropylene can be made with 99% of the current polymer and 1% of the additive.

This various components will have different durometer hardness of the fabric, straps, handles, buckles, fasteners, etc. Each of these components materials will have the additive added to their material. However, it's important to note that the actual hardness can vary depending on the specific manufacturing processes and additives used in the production of each material. Different grades and formulations can result in variations in hardness, even within the same category of material. In the embodiments of the invention, it is important that the hardware like buckles, fasteners, etc. is much more rigid than the fabric so that the hardware may firmly hold the support surface in place.

The various components will have different durometer hardness between the fabric of the straps, tubing, and the hardware. Each of these components materials will have the additive added to their material. However, it's important to note that the actual hardness can vary depending on the specific manufacturing processes and additives used in the production of each material. Different grades and formulations can result in variations in hardness, even within the same category of material. In the embodiments of the invention, it is important that conectors are much more rigid than the fabric of the mattress. For example, hardware, i.e. connectors will range between Shore D 45 to Shore D80 with a desired range of Shore D 50 to Shore D80 and a most desired Shore D 70 wherein in this range it will strike a balance between flexibility and stiffness. While offering a good durability and still maintaining some flexibility, making them suitable for various hardware applications.

The fabric is softer and more pliable and would be on the Shore A scale which is used to measure the hardness of soft pliable material. Shore A 30 to Shore A 70, the most desirable is Shore A 60 to Shore A 70 with a Shore A of 65. An mattress with durometer values in this range are very soft and pliable. Nylon parts with such hardness values are often used in applications where the material needs to be gentle against the skin. Parts with durometer values in this range are considered relatively soft and flexible. They offer good flexibility, drape, and conformability, making them suitable for a patient to be transported on.

In one embodiment, the invention discloses that although Nylon (polyamide) or Polyester Fabric or a nonwoven polypropyle) is the desired material in combination with a biodegradable additive, an aspect disclosed in embodiments of the invention are hospital disposables made of one or more non-biodegradable polymer (e.g. Nylon or Polyester Fabric) and an additive comprising a blend or copolymer of (1) a first polymer selected from a poly-lactic acid (PLA) and/or a polyhydroxyalkanoates (PHA) and (2) a second polymer which is a poly-terephthalate.

In one embodiment, the non-biodegradable polymer is present in the hospital disposable matter at a concentration of 90-99.9 wt. %, at a concentration of 95-99.5 wt. %, at a concentration of 98-99.5 wt. % or at a concentration of 99 wt. %, with the mass balance being the additive. In such embodiments, the additive is present at a concentration of 0.1-10 wt. %, 0.5-5 wt. %, 0.5-2 wt. % and 1% wt. %, respectively.

A further aspect of the embodiments is an additive for use with nylon comprising a blend of 30-70 wt. % poly-L-lactic acid and 30-70 wt. % poly-terephthalate, or an additive for use with polypropylene comprising a blend of 40-60 wt. % poly-L-lactic acid and 40-60 wt. % poly-terephthalate, or an additive for use with polypropylene comprising a blend of 45-55 wt. % poly-L-lactic acid and 45-55 wt. % poly-terephthalate, or an additive for use with polypropylene comprising a blend of 49-51 wt. % poly-L-lactic acid and 49-51 wt. % poly-terephthalate.

Another aspect is a method of making a hospital disposable comprising the steps of: blending polypropylene with an additive of poly-L-lactic acid and a poly-terephthalate; and at least one of extruding, molding and forming a hospital disposable from the blend.

Another embodiment of the invention would include adding an antimicrobial to the manufacturing process. The most common additives used to manufacture antimicrobial plastics include various isothiazolinone treatments, zinc pyrithione, thiabendazole, and silver antimicrobial products. Each active ingredient has its strengths and weaknesses.

The examples provided herein does not mean to limit or define the scope of the invention, and should not be construed as a restriction on the possible variations or embodiments of an invention. The following examples are provided to illustrate but not to limit the invention.

### Examples of Various Embodiments are Provided Herein Below.

### Example 1: Process of Making a Nylon Fabric

99 wt. % nylon was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then formed into nylon fabric.

Nylon fabric is a synthetic fabric made from polyamide fibers, and its creation involves several steps which are described herein below:
1. Polymerization: The first step is to create the nylon polymer, which is achieved through a process called polymerization. The main raw materials used in the polymerization of nylon are petroleum-derived chemicals, such as adipic acid and hexamethylenediamine. These chemicals react together under controlled conditions to form a liquid polymer called nylon salt.
2. Condensation: The nylon salt is then heated to high temperatures, causing it to undergo a condensation reaction. During this reaction, the nylon salt transforms into a solid nylon polymer. The nylon polymer is usually in the form of chips or pellets at this stage.
3. Melting and Extrusion: The solid nylon polymer is then melted and forced through spinnerets, which are small metal plates with tiny holes. The shape and size of the holes in the spinnerets determine the thickness and texture of the resulting nylon fibers. As the molten nylon polymer is extruded through the spinnerets, it solidifies and takes the form of long, continuous filaments.
4. Drawing: After extrusion, the nylon filaments are stretched or drawn to align the molecules and improve the strength and durability of the fabric. Drawing also affects the final texture of the fabric, making it smoother and more uniform.
5. Cooling and Solidification: The drawn nylon filaments are rapidly cooled to solidify them in their stretched state. This process helps to lock in the molecular alignment achieved during drawing.
6. Crimping (Optional): In some cases, the nylon filaments are crimped to introduce texture and bulkiness to the fabric, which is desirable for certain applications.
7. Spinning into Yarn: The solidified nylon filaments are then wound onto bobbins to form yarn. The yarn can be used in its pure form or blended with other fibers to create various types of nylon fabrics with different characteristics.
8. Weaving or Knitting: The nylon yarn is then woven or knitted into fabric on industrial looms or knitting machines. The weaving or knitting process creates the final fabric with the desired pattern and texture.
9. Finishing: After weaving or knitting, the nylon fabric undergoes various finishing processes to enhance its properties, such as dyeing, printing, and coating to make it water-resistant or flame-retardant. A biodegradable polyurethane and/or biodegradable water-resistant coating may be sprayed thereon.

The resulting nylon fabric is lightweight, strong, abrasion-resistant, and has excellent elasticity, making it suitable for use with a lateral transfer support surface.

### Example 2: Process of Making Polypropylene Buckles

99% polypropylene beads mixed with 1% additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then formed into polypropylene buckles.

Polypropylene buckles are formed through several steps which are described herein below:

Material Preparation: Polypropylene pellets or granules are selected as the raw material. These pellets are fed into the injection molding machine's hopper along with the additive.
Heating and Melting: The polypropylene pellets are heated and melted within the injection molding machine's barrel. The temperature is carefully controlled to ensure proper melting without causing degradation of the plastic material.
Injection: Once the polypropylene is molten, it is injected under high pressure into the mold cavity using a reciprocating screw or a plunger. The mold is closed and clamped to prevent any leakage during the injection process.
Cooling and Solidification: The molten polypropylene quickly starts to cool down as it comes into contact with the mold surfaces. Cooling channels within the mold help expedite the cooling process. As the plastic cools, it solidifies and takes on the shape of the buckle.
Mold Opening and Ejection: Once the polypropylene has solidified, the mold is opened, revealing the molded buckle. Ejection pins or mechanisms help push the buckle out of the mold.
Trimming and Quality Control: Any excess material or flash around the edges of the buckle is trimmed off. The buckle is then inspected for defects, proper dimensions, and overall quality. This step ensures that the final product meets the required specifications.

### Example 3: Process of Making Nylon Straps

99 wt. % nylon was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then woven into nylon straps using the conventional method in the field.

The hardware produced by Example 2 and Example 3 were then assembled onto the main body fabric of Example 1 to provide a biodegradable hardware.

### Biodegradation Testing

The injection molded plastic was tested under standard ASTM D5511. This test method covers the determination of the degree and rate of anaerobic biodegradation of plastic materials in high-solids anaerobic conditions. The test materials are exposed to a methanogenic inoculum derived from anaerobic digesters operating only on pretreated household waste. The anaerobic decomposition takes place under high-solids (more than 30% total solids) and static non-mixed conditions. This test method is designed to yield a percentage of conversion of carbon in the sample to carbon in the gaseous form under conditions found in high-solids anaerobic digesters, treating municipal solid waste.

Anaerobic digested sewage sludge was mixed with household waste. To make the sludge adapted and stabilized during a short post-fermentation at 53° C., the sludge was pre-incubated (one week) at 53° C. This means that the concentrated inoculum was not fed but allowed to post ferment the remains of previously added organics allowing large easily biodegradable particles were degraded during this period and reduce the background level of biogas from the inoculums itself.

A sample of the anaerobic digested sewage sludge was analyzed for pH, percent dry solids, and volatile solids, as well as, the amount of CO2 and CH4 evolution during the testing. Table 1 lists the results of this initial testing.

Inoculum Medium: Remove enough inoculum (approximately 15 kg) from the post-fermentation vessel and mix carefully and consistently by hand in order to obtain a homogeneous medium. Test three replicates each of a blank (inoculum only), Positive control (Reference material) (thin-layer chromatography cellulose), negative control (optional), and the test substance being evaluated.

Manually mix 1000 g wet weight (at least 20% dry solids) of inoculum in a small container for a period of 2 to 3 min with 15 to 100 g of volatile solids of the test substance or the controls for each replicate. For the three blanks containing inoculum only, manually mix 1000 g of the same inoculum in a small container for a period of 2 to 3 min with the same intensity as was done for the other vessels containing test substance or controls. Determine the weight of the inoculum and test substance added to each individual Erlenmeyer flask accurately. Add the mixtures to a 2-L wide-mouth Erlenmeyer flask and gently spread and compact the material evenly in the flask to a uniform density.

After placing the Erlenmeyer flask in incubator, connect it with the gas collection device. Incubate the Erlenmeyer flasks in the dark or in diffused light at 52° C. (±2° C.) for thermophilic conditions, The incubation time shall be run until no net gas production is noted for at least five days from both the Positive control (Reference material) and test substance reactors. Control the pH of the water used to measure biogas production to less than two by adding HCl.

The most important biochemical characteristics of the inoculum such as pH, Volatile Fatty Acids, NH4+-N— and dry solids were studied.

TABLE 1
Results of Initial testing of the anaerobic digested sewage sludge

| Parameters | Requirement | Actual results |
|---|---|---|
| pH | 7.5 to 8.5 | 7.85 |
| Kjeldahl nitrogen | 0.5 to 2 g/kg wet weight | 1.46 |
| Dry Solids at 105° C. | >20% | 44.00 |
| Volatile Solids at 550° C. | Below 1 g/kg wet weight | 0.76 |

The biogas volume in the gas sampling bag was measured (Table-2). Presence of gas in the gas collector of Positive control indicated that the inoculum was viable and gas displacement was observed both in Positive control and Test Sample.

ASTM D5511 states that for the test to be considered valid, the Positive control must achieve 70% within 30 days with deviation less than 20% of the mean between the replicates.

Positive control (Reference material) showed 71.03% on 27th day with less than 20% of the mean difference between the replicates.

The gas displacement observed after 90 days is as shown in the table below.

TABLE 2
Biogas volume of the evolved gas during
the biodegradation process at 90 days

| Biodegradation | Total Volume |
|---|---|
| Test | 90 days (mL) |
| Inoculum | 2840 |
| Positive control (Reference material) | 10100 |
| Bioplastic | 6500 |

The percent biodegradation of Positive control (and Test sample was calculated by the measured cumulative carbon dioxide and methane production from each flask after subtracting carbon dioxide evolution and methane evolution from the blank samples at the end 90 days of testing. Calculations were based on Total Organic Carbon obtained of both Positive control and Test sample.

TABLE 3a
Percentage biodegradability of Test sample
with respect to Positive control

| | | Positive control | Biodegradable |
|---|---|---|---|
| | Inoculum | (Reference | plastic |
| Cellulose. Group | control | material) | Sample |
| Weight | 1000 ml | 10.2305 g | 10.1335 g |
| Total volume (ml) | 2840.00 | 10100.00 | 6500.00 |
| % CH4 | 13.20 | 44.90 | 21.80 |
| Volume of CH4 | 374.88 | 4534.90 | 1417.00 |
| (ml) | | | |
| weight of CH4 (g) | 0.2459 | 2.9749 | 0.9296 |
| % CO2 | 15.70 | 45.50 | 24.20 |
| Volume of CO2 | 445.88 | 4595.50 | 1573.00 |
| (ml) | | | |
| Weight of CO2 (g) | 0.8828 | 9.0991 | 3.1145 |
| Total weight of | 0.4228 | 4.6879 | 1.5381 |
| carbon in grams | | | |
| Theoretical weight | — | 4.3040 | 8.4999 |
| of carbon in grams | | | |
| (Ci) | | | |
| Biodegradation | — | 0.99097 | 0.13121 |
| % Biodegradation | — | 99.10 | 13.12 |

Based upon the above, the biodegradable polymer/plastic showed a 13.12% biodegration over 90 days. This satisfies the ASTM D5511 standards for biodegradation.

What is claimed is:

1. A lateral transfer support surface, comprising:
flexible material sheets sealed to one another to form at least one chamber, at least one of the flexible material sheets having air exit holes therein, said flexible material sheets comprised of:
a polymer selected from a group consisting of polyamide, polyester, polyethylene (PE), polypropylene (PP), polyvinylchloride (PVC) and combinations thereof; and
an additive comprising a blend or copolymer of (1) a first polymer selected from a poly-lactic acid (PLA), a polyhydroxyalkanoates (PHA) or a combination thereof and (2) a second polymer which is a poly-terephthalate.

2. A lateral transfer support surface comprising:
flexible material sheets sealed one another to form at least one chamber, at least one of the flexible material sheets having air exit holes therein, said flexible material sheets comprised of:
a non-biodegradable polymer selected from a group consisting of polyamide, polyester, polyethylene (PE), polypropylene (PP), polyvinylchloride (PVC) and combinations thereof; and
an additive comprising a blend or copolymer of (1) a first polymer selected from a poly-lactic acid (PLA), a polyhydroxyalkanoates (PHA) or a combination thereof and (2) a second polymer which is a poly-terephthalate;
a polyurethane coating on the flexible sheets, said polyurethane coating having an additive comprising a blend or copolymer of (1) a first polymer selected from a poly-lactic acid (PLA), a polyhydroxyalkanoates (PHA) or a combination thereof and (2) a second polymer which is a poly-terephthalate.

3. A biodegradable lateral transfer support surface comprising:
a. fabric; and
b. hardware, wherein the fabric and hardware are both made from a material comprised of:
i. a non-biodegradable polymer selected from a group consisting of polyamide, polyester, polyethylene (PE), polypropylene (PP), polyvinylchloride (PVC) and combinations thereof; and
ii. a biodegradable additive,
wherein the fabric has a first hardness on the durometer scale and the hardware has a second hardness on the durometer scale, different than the first hardness on the durometer scale.

4. The biodegradable lateral transfer support surface system of claim 1, wherein the main body and hardware are made of the same material.

5. The biodegradable lateral transfer support surface of claim 1, wherein the main body fabric has a hardness of Shore A 50 to Shore 70 hardness.

6. The biodegradable lateral transfer support surface of claim 1, wherein the main body fabric hardware has a Shore D 45 to Shore D60 hardness.

7. A biodegradable lateral transfer support surface comprising:
a. a main body selected from polyamide or polyester;
b. inflatable chambers comprise a non-biodegradable polymer selected from a group consisting of polyethylene (PE), polypropylene (PP), polyvinylchloride (PVC) and combinations thereof;
c. straps and handles selected from a group consisting of nylon, polyester, polyethylene (PE), polypropylene (PP), polyvinylchloride (PVC) and combinations thereof;
d. fasteners selected from a group consisting of nylon, polyester, polyethylene (PE), polypropylene (PP), polyvinylchloride (PVC) and combinations thereof; and
e. an additive added to each of the materials in a-d comprising a blend or copolymer of (1) a first polymer selected from a poly-lactic acid (PLA), a polyhydroxyalkanoates (PHA) or a combination thereof and (2) a second polymer which is a poly-terephthalate.

8. The biodegradable lateral transfer support surface of claim 5, wherein wherein the non-biodegradable polymer is present in a concentration from 90-99.9 wt. %, the additive is present in a concentration from 0.1-10 wt. % and the additive has 30-70 wt. % of the first polymer and 30-70% of the second polymer.

9. The biodegradable lateral transfer support surface of claim 1, further comprising:
an antimicrobial additive.

10. The biodegradable lateral transfer support surface of claim 7, wherein the antimicrobial is selected from isothiazolinone treatments, zinc pyrithione, thiabendazole, and silver.

11. The biodegradable lateral transfer support surface of claim 5, wherein the main body fabric and hardware have different durometer hardnesses.

12. A method comprising:
providing a polymer selected from a group consisting of nylon, polyester, polyethylene (PE), polypropylene (PP), polyvinylchloride (PVC) and combinations thereof;
providing an additive comprising a blend or copolymer of (1) a first polymer selected from a poly-lactic acid (PLA), a polyhydroxyalkanoates (PHA) or a combination thereof and (2) a second polymer which is a poly-terephthalate; and
wherein the non-biodegradable polymer is present in a concentration from 90-99.9 wt. %, the additive is present in a concentration from 0.1-10 wt. % and the additive has 30-70 wt. % of the first polymer and 30-70% of the second polymer;
blending the polymer and the additive and forming a fabric;
cutting the fabric into material sheets;
perforating at least one sheet of the sheets;
sewing the sheets into a lateral support surface with chambers and an opening to provide constant air flow.

13. The method of claim 12, further comprising coating the lateral support surface with a a polyurethane coating on the material sheets, said polyurethane coating having an additive comprising a blend or copolymer of (1) a first polymer selected from a poly-lactic acid (PLA), a polyhydroxyalkanoates (PHA) or a combination thereof and (2) a second polymer which is a poly-terephthalate.

14. The composition of matter as recited in claim 10, wherein the non-biodegradable polymer is present in a concentration from 95-99.5 wt. % and the additive is present in a concentration from 0.5-5 wt. %.

15. The composition of matter as recited in claim 10, wherein the non-biodegradable polymer is present in a concentration from 98-99.5 wt. % and the additive is present in a concentration from 0.5-2 wt. %.

16. The composition of matter as recited in claim 10, wherein the non-biodegradable polymer is present in a concentration of 99 wt. % and the additive is present in a concentration of 1 wt. %.

17. The composition of matter as recited in claim 10, wherein the poly-terephthalate is a random block copolymer of: 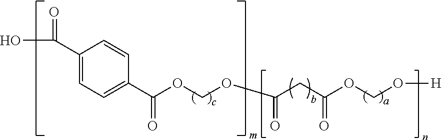
wherein a and c are independently selected integers from 1 to 6, b is an integer from 1 to 8, and m and n are numbers that are within 30% of one another.
